(19) United States
(12) Patent Application Publication
(10) Pub. No.: US 2023/0342726 A1
(43) Pub. Date: Oct. 26, 2023

(54) METHOD FOR SCHEDULING LIVE CALL WITH UNAVAILABLE SERVICE PROVIDER

(71) Applicant: TASKHUMAN, INC, Saratoga, CA (US)

(72) Inventors: Kartik Thumbavanam PADMANABHAN, Fremont, CA (US); Daniel MAZZELLA, West Caldwell, NJ (US); Ravi SWAMINATHAN, Palo Alto, CA (US); Allen SAMUELS, San Jose, CA (US)

(73) Assignee: TASKHUMAN, INC, Saratoga, CA (US)

(21) Appl. No.: 17/777,965

(22) PCT Filed: Oct. 21, 2019
(86) PCT No.: PCT/US19/57299

§ 371 Date: May 18, 2022

(51) Int. Cl. G06Q 10/1093

(52) U.S. Cl. CPC G06Q 10/1095

(57) ABSTRACT

System and method for a consumer to arrange a live bi-directional audio/video feed to a previously unknown service provider either by selecting from a set of dynamically determined categories or through natural language search techniques. Service providers have previously indicated their availability which the system uses to select and present to consumers.

### BACKGROUND AND SUMMARY OF THE INVENTION

In the present-day world, people rely on search engines and various review sites for numerous reasons. Whether it involves buying a house, starting a new business, working towards retirement or losing weight, people constantly use search engines to seek knowledge and advice. With the constant evolution of the digital age, web search engines and review sites inundate consumers with a deluge of information. For example, a simple query on ‘losing weight’ results in millions of hits on search engines or hundreds of options in a review site, some of which may not be suitable for the knowledge seeker's particular situation or may be incomplete, leaving the knowledge seeker with further follow-up questions. In situations like these, it's more effective to use a human provider (or a Subject-matter expert) to help educate knowledge seekers with the answers to their questions.

With the ever-growing popularity of online marketplaces, it's now possible for the knowledge seeker (hereinafter “consumer”) to instantly get connected to a subject matter expert (hereinafter “service provider”) to talk about and act on a particular topic that the consumer is interested in. However, most of these marketplaces still have a lot of friction in serving consumers and providers alike. For example they only serve only a particular category of Human providers (e.g. doctors, mechanics, psychics, etc.) and connecting the consumers and the service providers are cumbersome and involve a tedious process of consumers having to fill in a series of questions presented to them in order to meet the requirements to direct the consumer to the relevant provider.

Currently, there is no online marketplace for service providers that not only connects consumers to the service providers via bi-directional video/audio through a single input mechanism or directly from a topical search engine that intakes consumer queries and directs consumer to the relevant service providers, but also provides a vehicle to add new topics of expertise as the service provider knowledge base and the number of service providers grows. Humans are continuous learners and even experts update their knowledge with the latest findings in their field and other information close to their field of expertise. For example, a Dietician keeps up to date with the current state of his/her art, the practices and emerging sciences related to ‘Diets and Nutrition’ as well as supplementary topics such ‘Stress’ or ‘Physical Exercise’. A Soccer coach keeps up to date on all major happenings around his/her sport as well as information about other sports/activities that his/her players might benefit from trying. Once the service provider feels comfortable claiming expertise in a new topic, they should be able to add that to their profiles with ease. For this, there exists a need for an online repository that's current with the present state of expertise for human service providers in the world. Aspects of the present disclosure fill this need.

Additionally or alternatively, a current problem among conventional sites that offer expert advice for a variety of topics is that such conventional sites do not allow for immediate or approximately immediate real-time interaction (e.g., via live video and/or audio feed) in bi-directional communication. For example, a host of intermediary steps may first be required before any interaction between the consumer and the service provider is allowed, and the interaction in many cases may not be live interaction (e.g., bi-directional video and/or audio). The intermediary steps may include login steps, account generation, upfront payments, agreement to terms and conditions, scheduling an appointment, sending a request to the service provider through mechanism such as email or instant messaging, sorting through a myriad of service providers and selecting a service provider, copying and pasting and/or dialing a number in a profile of the service provider, and sorting or scrolling through service provider categories (e.g., topics in which the service providers are experts). The intermediary step(s) required by conventional sites may deter many consumers. None of the conventional sites that offer expert advice include an interface that allows for direct subsequent follow on interaction via a live bi-directional video/audio feed as well as a mechanism to exchange information in the form of images, files, and other computer based information exchange mechanisms in response to a consumer providing a single consumer input at a consumer interface of a client device.

Aspects of the present disclosure may be directed to solving the above-described example problems. For example, a technical solution rooted in computer technology and the internet may include linking within a time frame (e.g., about 5 seconds, about 60 seconds, about 120 seconds, about 180 seconds, etc.) the consumer to the service provider in response to the consumer selecting the service category without any intermediary steps. The linking may include establishing an internet protocol (IP) packet based communication connection, including a bi-directional video and/or audio communication connection, between the client device of the consumer and a device of the service provider determined to link to the client device, the IP packet based transmission connection established by a system that hosts the database of service categories, wherein the system includes the IP addresses of the client device and the device of the service provider to establish the communication connection and the client device and the device of the service provider are unware of each other's IP addresses before the determination of the service provider.

Part of the technical solution of quickly linking the consumer to the service provider by linking as described above may include managing in real time the availability of the service providers in a number of service categories. The managing in real time may include determining whether at least one service provider in each service category has sent an availability input indicating if and when the service provider is available to engage the consumer, wherein the determining includes counting a number of service providers that currently indicate availability to engage the consumer and/or determining the amount of expertise a provider may have on the subject matter, the supply and demand ratio of the user and the service providers on the discovery topic at that point of time. Additionally or alternatively, the managing in real time may include in response to the determining, updating the real-time status indicator of each service category to reflect a current availability of one or more service providers in the service category by re-rendering at the client device the corresponding real-time status indicator, wherein a status change for any service category constitutes a change in availability to engage the consumer as follows: (i) changing from (status A) at least one service provider in the service category currently provides an availability input that indicates an availability to engage the consumer to (status B) no service provider in the service category currently provides an availability input that indicates an availability to engage the consumer, or (ii) changing from (status B) no service provider in the service category currently provides an availability input that indicates an availability to engage the consumer to (status A) at least one service provider in the service category currently provides an availability input that indicates an availability to engage the consumer. With the help of this availability input, the user can engage with the service provider on that category at a later point of time.

Also part of the technical solution includes pre-selecting a group of service categories and corresponding service providers that are custom tailored to the consumer to service provider pairing, which makes the quick linking between the consumer and the service provider more effective and/or more efficient for the consumer and service provider. Not only are intermediary steps removed for the consumer, but they are intelligently performed (e.g., by an artificial intelligence engine). The pre-selecting may include scraping of consumer or service provider data from one or more of: a social media posting of the consumer or service provider, a website of the consumer or service provider, a publication of the consumer or service provider, the consumer's or service provider's pictures on a social media site or client device, a location of the consumer or service provider, or a preferred language of the consumer or service provider. Additionally or alternatively, the pre-selecting may include analyzing the consumer data to correlate a relationship between the consumer data and the database of service categories. Based on the correlated relationship being above a threshold correlation, the system of the present disclosure may select the plurality of service categories for the consumer.

Also part of the technical solution to quickly link the consumer to the service provider includes adapting to real-time changes in availability of service providers and consumer. For example, when the consumer selects a service category, availability of one or more service providers may have simultaneously changed or just prior (e.g., about 0.1 seconds—about 10 seconds prior) to the consumer input selecting the service category. Other variables to a working technical solution also may be addressed. Thus, when the consumer selects a service category, the system of the present disclosure may determine which of the one or more service providers to link with the consumer based on one or more of the scraped consumer data, a search query, the availability inputs received from the one or more service providers between a most recent re-rendering of the real-time status indicator and receiving the consumer input, and a priority relationship among the one or more service providers. For example, the availability inputs from the one or more service providers received from the one or more service providers between a most recent re-rendering of the real-time status indicator and receiving the consumer input may change which service providers are available to engage the consumer such that either more or fewer service providers are actually available to engage the consumer. Additionally or alternatively, the priority relationship among the one or more service providers may determine an order to which service providers are linked with consumers, the priority relationship based on one or more of consumer ratings of the service provider, a referral of a service provider by a consumer or another service provider, a subscription tier paid by the service provider or consumer that includes a certain order or threshold ranking, and a quality control assessment of the service provider that includes a skill ranking within the service category.

Also a part of the technical solution is to handle the situation where the selected service category with one or more service providers has no current availability. In an alternate embodiment, it could arise that there are service providers currently available, but the consumer has a preference for a service provider that is not currently available, creating essentially the same “no current availability” situation. One mechanism to handle this situation is for the system to display the future availability of the one or more service providers, allowing the consumer to select one. This could be done on a category basis where the system displays the aggregated availability of the one or more service providers or it could be done on an individual provider basis where the system displays the future availability of each individual provider. In either case the consumer is invited to select one of the future availabilities, creating an appointment.

However, the future availability of the one or more service providers (whether aggregated or not) may not be acceptable to the consumer for several possible reasons. One reason is that the consumer may not be available during one of the future availabilities of the one or more service providers. Another reason could be a preference for a particular service provider who is not currently available. In this case the consumer may ask the system to make a provider available at one or more specific times. The request might be generic or specific. In the generic case the consumer is willing to accept any provider that services the selected service category. In the specific case, the provider designates one or more specific providers that are desired at the one or more specified times.

A future availability request may have a differentiated cost structure associated with it. In one embodiment, a consumer could choose to accompany the request with a specific financial incentive (a tip) to entice a provider to create the additional availability. In another embodiment, future availability requests might have a standard fee imposed on them by the system. In another embodiment, providers bid for the additional business, resulting in a variable cost (possibly marked up by the system) to the consumer. The consumer might choose to reject the additional variable cost, effectively rescinding the request for future availability or wait for a potentially cheaper provider. In another embodiment, the consumer might provide a cost limit to the request, implicitly signaling the acceptance of a fee that is below the set amount.

Also part of the technical solution is to maintain an ever growing list of service categories that is in sync with the current state of the world. For example, if a new immigration law is included the system is capable of identifying the new law and create a topic around it and make a dynamic availability on all the immigration law service providers who either spoke about the law in the system (during past user engagements) or updated about it in their social media feed. The system may also rank these providers based on how much these providers spoke about it on their social media feed and direct those service providers first to interact with the users looking out for the topic. The system may also identify these topics from the footprints obtained from the user's social media feed or their past interaction with the system and may present these newer categories to the users.

Also part of the technical solution is to go as granular as possible while presenting these discover topics to the user. The system is capable of not only identifying experts and displaying discover topics on higher level categories like Bodybuilding, Yoga etc. but also go more granular and present them as discover topics. For example a discover topic could be a Bodybuilding supplement (instead of bodybuilding) or Iyengar Yoga and identify providers in the system who are subject matter experts in bodybuilding supplements or Iyengar Yoga respectively. The subject matter expertise may again be determined by the providers past engagement with the system and the amount of information extracted from the providers social media feeds or other public or private data shared by service provider or collected about service provider from third party companies and may rank them appropriately. The system may maintain an internal graph structure to define various parent and child relationships (for example Asana yoga being a child of parent category Yoga) and maintain a data structure containing service providers in all of those parent-child categories.

Based on one or more aspects of the technical solution, which is rooted in computer technology and the internet, introduced above and further articulated below, the example problems described in this disclosure may be addressed.

### BRIEF DESCRIPTION OF THE DRAWINGS

FIG. 1 shows an overview of the current system

FIG. 2 shows an overview of the method of the current system.

FIG. 3 shows the category matching process.

FIG. 4 shows the process to render categories on the client device.

FIG. 5 shows how service providers are selected for a particular category.

FIG. 6 shows the processing of a call.

FIG. 7 shows an example of a category selection screen after processing a query.

FIG. 8 shows the query expansion process.

FIG. 9 shows the mapping table from spot to category.

FIG. 10 shows the organization of the various corpuses used by the search engine.

FIG. 11 shows the process of scheduling an appointment.

FIG. 12 shows the process of auctioning for future availability.

### DETAILED DESCRIPTION OF THE INVENTION

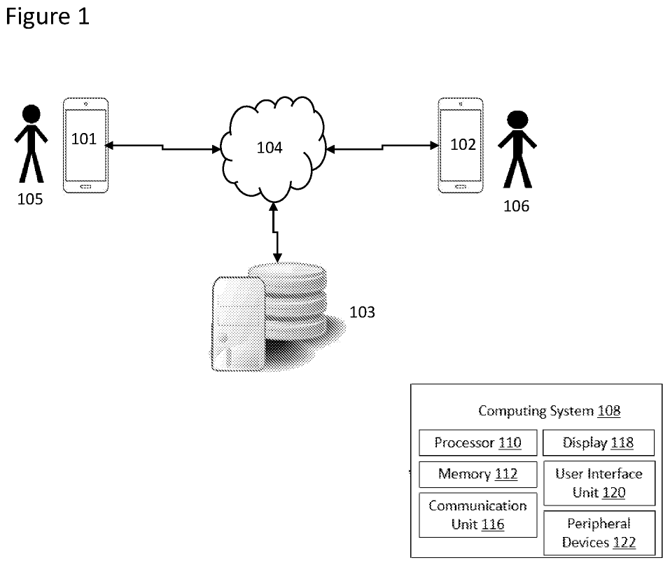

FIG. 1 shows an overview of the current system. Consumer 105 uses consumer computer system 101. Service provider 106 uses service provider computer system 102. Consumer computer system 101, service provider computer system 102, and server computer system 103 are communicatively coupled through network 104. Each of consumer computer system 101, service provider computer system 102 and server computer system 103 are separate instances of computing system 108 with different amounts and types of each of the contained resources: processor 110, memory 112, communication unit 116, display 118, user interface unit 120 and peripheral devices 122 which are themselves operatively coupled. For example, server computer system 103 may not need display 118 or user interface unit 120 and thus they may not be present. In some embodiments network 104 is an Internet Protocol (IP) packet-based network. Each of the three computer systems (101, 102 and 103) are executing application programs (not shown) that contain the instructions to participate and perform the operations described herein. As is well-known, the three application programs could be instances of a common application program each configured with for the individual behavior or separate programs each dedicated to the relevant behavior. In the full system there are one or more consumers 105 each with a separate consumer computer system 101 and one or more service providers 106 each with a separate service provider computer system 102 (not necessarily the same number of each), but these are omitted for clarity.

For descriptive clarity, this specification will often refer to actions of computers systems where it is understood that these computer systems are operating according to the instructions of the corresponding application program.

Generally, the processor 110 may include any suitable special-purpose or general-purpose computer, computing entity, or processing device including various computer hardware or software modules and may be configured to execute instructions stored on any applicable computer-readable storage media. For example, the processor 110 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data.

Although illustrated as a single processor in FIG. 1, it is understood that the processor 110 may include any number of processors distributed across any number of networks or physical locations that are configured to perform individually or collectively any number of operations described in this disclosure. In some embodiments, the processor 110 may interpret and/or execute program instructions and/or process data stored in the memory 112. In some embodiments, the processor 110 may execute the program instructions stored in the memory 112.

For example, in some embodiments, the processor 110 may execute program instructions stored in the memory 112 that are related to, for example, managing in real time the availability of service providers, and/or may perform or direct the performance of the operations associated therewith as directed by the instructions. In these and other embodiments, instructions may be used to perform one or more operations of any method or figure of the present disclosure.

The memory 112 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 110. By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store particular program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 110 to perform a certain operation or group of operations as described in this disclosure. In these and other embodiments, the term “non-transitory” as explained in the present disclosure should be construed to exclude only those types of transitory media that were found to fall outside the scope of patentable subject matter in the Federal Circuit decision of In re Nujten, 500 F.3d 1346 (Fed. Cir. 2007). Combinations of the above may also be included within the scope of computer-readable media.

The communication unit 116 may include any component, device, system, or combination thereof that is configured to transmit or receive information over network 104. In some embodiments, the communication unit 116 may communicate with other devices at other locations, the same location, or even other components within the same system. For example, the communication unit 116 may include a modem, a network card (wireless or wired), an infrared communication device, a wireless communication device (such as an antenna), and/or chipset (such as a Bluetooth device, an 802.6 device (e.g., Metropolitan Area Network (MAN)), a Wi-Fi device, a WiMax device, cellular communication facilities, etc.), and/or the like. The communication unit 116 may permit data to be exchanged with any devices or systems described in the present disclosure.

The display 118 may be configured as one or more displays, like an LCD, LED, or other type of display. The display 118 may be configured to present video, text, numbers, consumer interfaces, and other data as directed by the processor 110.

The user interface unit 120 may include any device to allow a consumer to interface with the computing system 108. For example, the user interface unit 120 may include a mouse, a track pad, a keyboard, buttons, and/or a touchscreen, among other devices. The user interface unit 120 may receive input from a consumer and provide the input to the processor 110. In some embodiments, the user interface unit 120 and the display 118 may be combined.

The peripheral devices 122 may include one or more devices. For example, the peripheral devices may include a sensor, a microphone, and/or a speaker, among other peripheral devices. In these and other embodiments, the microphone may be configured to capture audio, e.g., audio from a consumer in the form of a search query. The speaker may broadcast audio received by the computing system 108 or otherwise generated by the computing system 108.

### System Overview

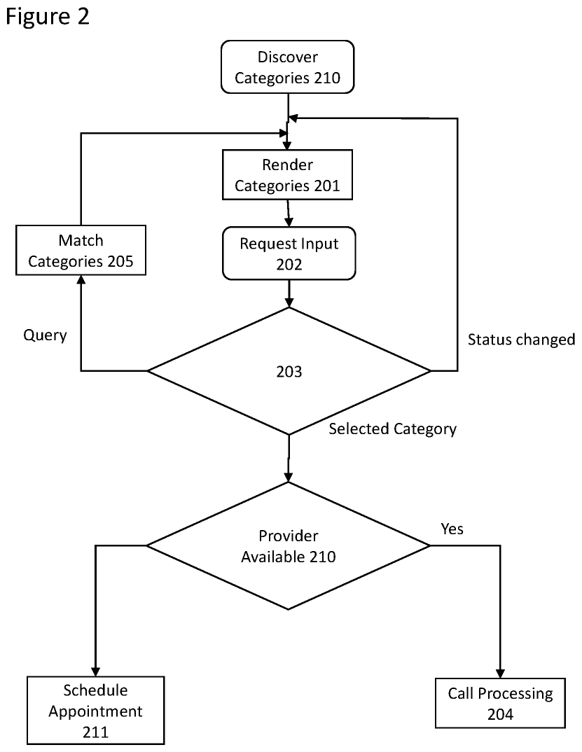

Turning to FIG. 2, an initial set of categories is determined by the discover categories step 210. Those categories are presented to consumer 105 by render categories step 201 (see below). Input is requested from consumer 105 in step 202.

If the consumer input is a search query then the match categories step 205 converts the query into the set of categories and the system proceeds to the render categories step 201.

While waiting for a consumer input the system may detect that a status has changed whereupon it will terminate waiting for consumer input and proceed to render categories step 201.

If the consumer input is the selection of a specific category, then the system determines if one or more providers are currently available in which case it proceeds with the call processing step 204. If no providers are currently available, then it proceeds with step Schedule Appointment 211.

### Match Categories (205)

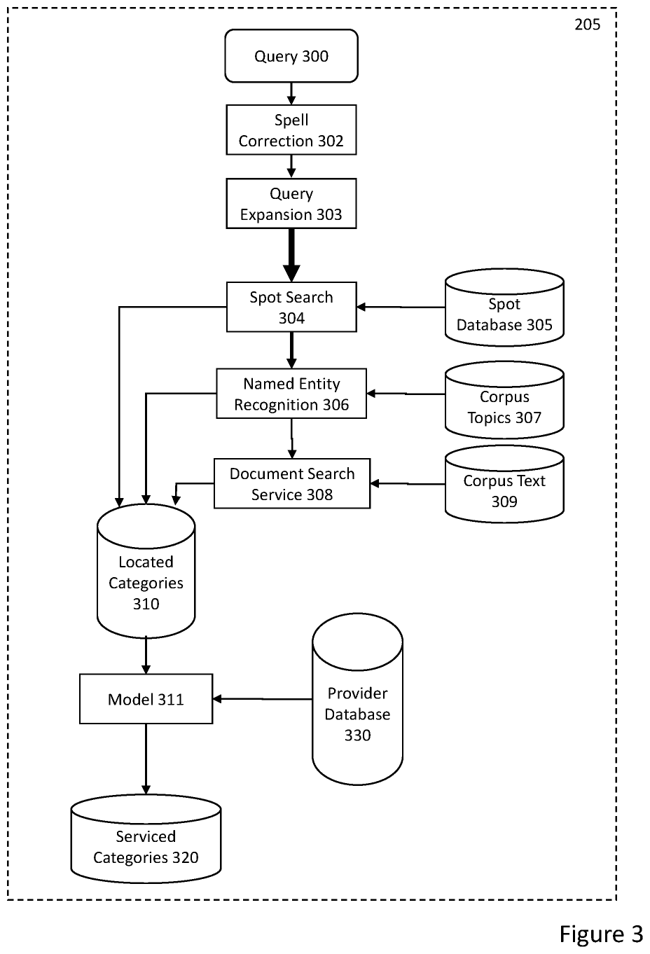

Match Categories step 205 is further described in FIG. 3. Query 300 received from request input step 202 is subjected to spell correction 302 using well-known techniques such as Levenshtein distance, Hamming distance, cosine distance, Jaro distance, q-gram, Jaccard distance, etc. Query terms that are declared incorrect may be replaced by corrected versions of these same terms.

During query expansion 303 the query is augmented by additional words that may include related words, root words, genus words, species words, etc. FIG. 8 shows an example of query expansion 303 wherein the original query for ‘cars’ is augmented by ‘automobile’ and ‘auto’ which are commonly used synonyms. While in many cases an augmented query may have only a single query term, this description will use the plural form in order to simplify the language and should be understood to include the case of a single query term.

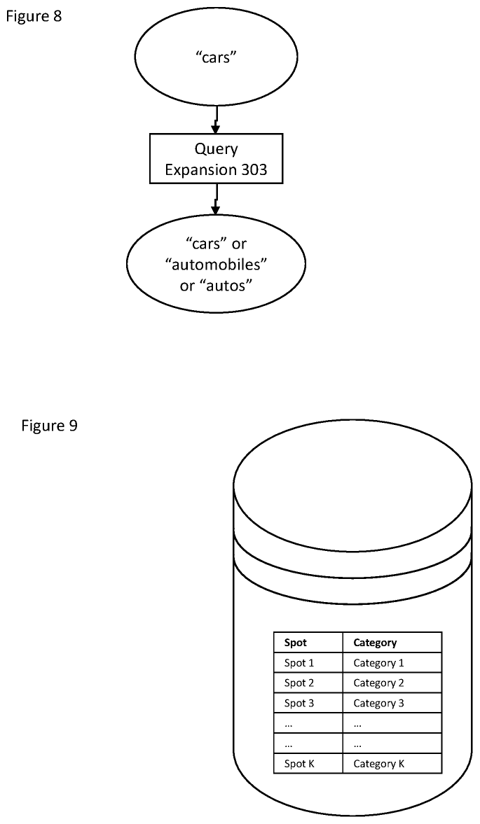

Spots are stored in spot database 305. Each spot in spot database 305 provides a mapping from an entity to a category as shown in FIG. 9. The terms of the augmented query are looked up in spot database 305 by considering query terms as entities. Query terms that are found in spot database 305 provide categories for those terms and are added to located categories 310. Query terms that are not present in spot database 305 are subjected to named entity recognition 306. Named entity recognition provides categories for query terms by locating the query terms in the corpus topics database 307 and adding those into located categories 310. Query terms which are not recognized by named entity recognition 306 are subjected to document search service 308. Document search service 308 provides categories for query terms by performing full text searches on corpus text 309. Query terms located by document search service 308 have their corresponding category added to located categories 310.

An example entry in spot database 305 might indicate that the term “Nagekomi” (Japanese judo term for repeated throwing practice) is a spot for the category “Judo”. Additional categories may be located through nearest neighbor techniques. For example, if “Nagekomi” (Japanese judo term for repeated throwing practice) is included as a spot for the category “Judo”, the final user recommendation for the user may include “Judo” and neighbors like “Uke”, “Aikido”, “Ninjutsu” and “Jujutsu”. These nearest neighbors can be found using natural language processing techniques that essentially maps each terminology in a corpus into a vector representation using algorithms like TFIDF (term frequency—inverse document frequencies, Doc2Vec, Word2Vec, Global Vectors (GloVe), etc.) and uses distance metrics like Hamming, Cosine or Manhattan distance to find the nearest neighbors.

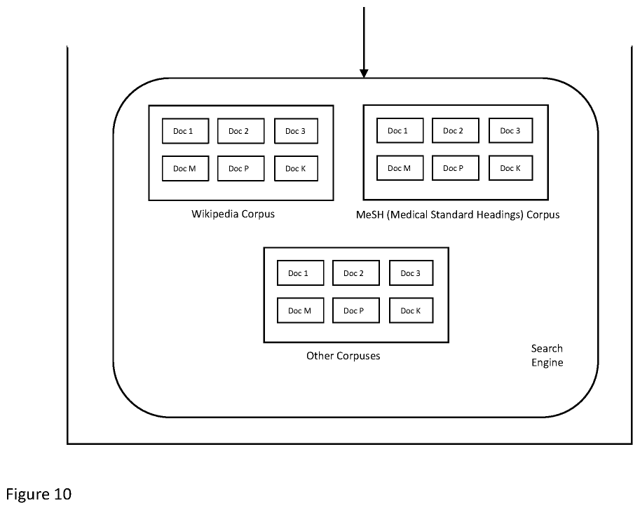

Corpus text 309 is composed of multiple authoritative corpuses one of which may be Wikipedia corpus, another may be a MeSH (Medical Standard Headings) corpus, and another may be another type of corpus as illustrated in FIG. 10. Corpus topics database 307 may be constructed by processing of Corpus text 309. Some corpuses may already have a topics database which would simplify the construction of corpus topics database 307.

Located categories 310 and provider database 330 are processed by model 311 to form a set of serviced categories 320 which is the subset of categories present in provider database 330 that are considered to be good matches for located categories 310. Model 311 may include one or more elements of machine learning, deep learning, natural language processing (NLP), autonomous databases, Bayesian statistics, recommendation systems, search relevance (e.g., modeling and/or vectorization), time-series forecasting, etc. alone or in combination.

The entries in spot database 305 are generated by processing of content from sources other than corpus text 309. Sources of content include: a social media posting of the consumer or service provider, a website of the consumer or service provider, a publication of the consumer or service provider, the consumer's or service provider's pictures on a social media site or client device, etc. Processing is done using one or more elements of machine learning, deep learning, natural language processing (NLP), autonomous databases, Bayesian statistics, recommendation systems, search relevance (e.g., modeling and/or vectorization), time-series forecasting, etc. alone or in combination.

### Render Categories (201)

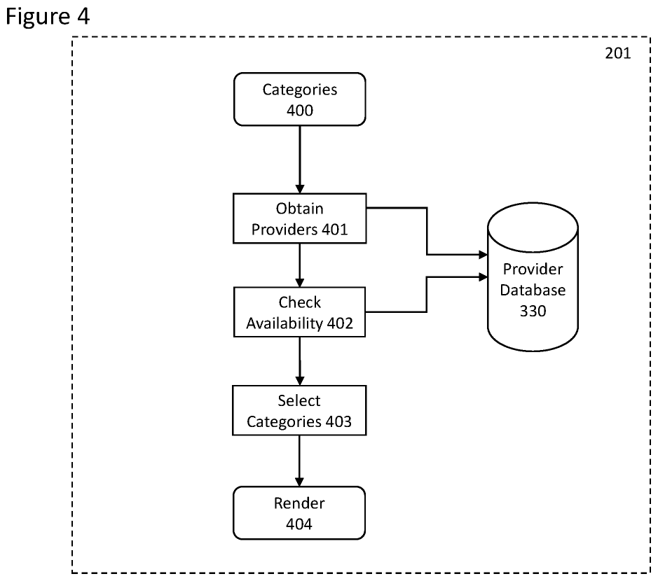

Render Categories step 201 is further described in FIG. 4. Each category of categories 400 is used to access provider database 330 to obtain the list of service providers for that category. In check availability step 402 each provider is determined to be currently available or not. Service provider availability information may be stored in provider database 330.

Select categories step 403 makes the final determination of which categories will be displayed and in what order. Many criteria can be considered for this final determination including: quantity of providers for a category, number of currently available providers for a category, quality of match of a category to the original query, subscription level of a provider, skill rating of a provider, location of a provider, language of a provider, etc.

Render 404 takes categories selected and prioritized by select categories 403 and generates a selection display upon consumer computer system 101. In a preferred embodiment, if categories 400 comes from match queries 205 then the display may also show the original query terms and/or the augmented query terms. In a preferred embodiment, each displayed category is decorated so as to indicate whether a provider is currently available in that category. In some embodiments additional information could be displayed for each category, for example: actual number of available providers, identification information for providers, etc. The selection display may itself be divided into groups of categories to indicate additional information like: group availability, quality of category match to a query term, etc.

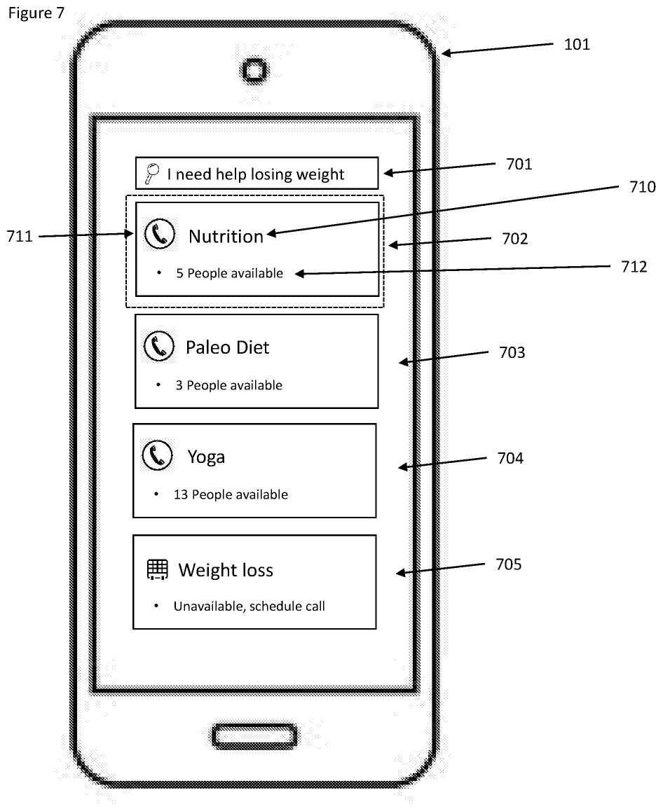

FIG. 7 shows an example output of render 404. Element 701 is a well-known search bar which in this example repeats the original query “I need help losing weight”. Visual element 702 corresponds to a category. Element 710 is the text of the name for the category. Element 711 is an icon indicating whether one or more providers are currently available. Element 712 provides additional descriptive text (in this example, the actual number of available providers is available). Elements 703 and 704 are similar to Element 702 except that the category name and additional descriptive text are different. Element 705 provides an example of a recognized category for which no providers are currently available. The calendar icon and descriptive text inform the user while no provider is currently available a calendaring mode of the program could be invoked to schedule a future call. In a preferred embodiment the text of the above elements is rendered in the language of consumer 105.

### Select Provider (601)

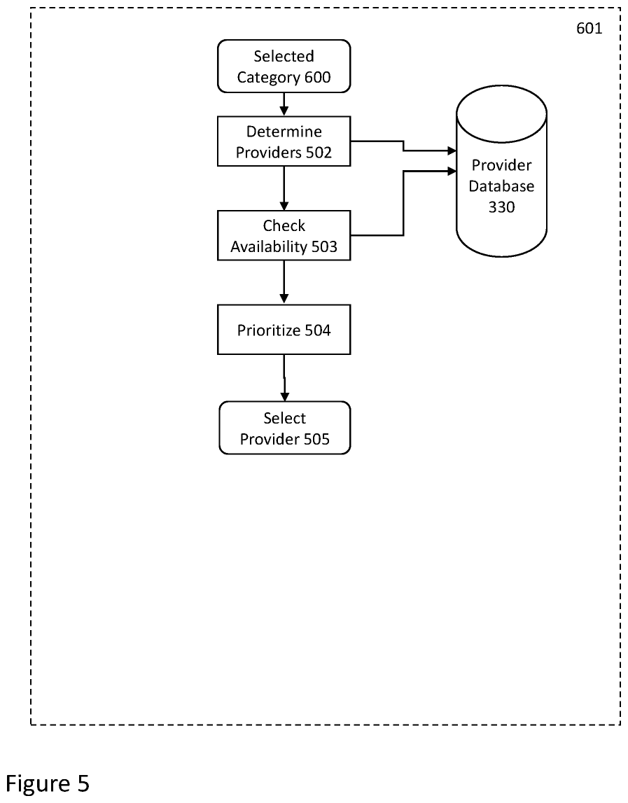

FIG. 5 shows the process of selecting an individual provider. Determine providers step 502 looks up selected category 600 in provider database 330 to obtain a list of providers that service selected category 501. Check availability step 503 determines the current availability of each provider using information from provider database 330 and potentially other information (see below on determining provider availability).

Prioritize step 504 ranks the providers. Many criteria can be used alone or in combination, e.g., provider subscription level, consumer subscription level, quality of provider, whether this consumer has previously been connected to this provider, query terms, timely responsiveness (e.g., connecting to bi-directional audio/video feeds), provider skills with respect to the query terms and/or selected category, etc. Provider quality and/or skills may be determined according to an analysis of their social media, resumes, published articles, etc.

### Call Processing (204)

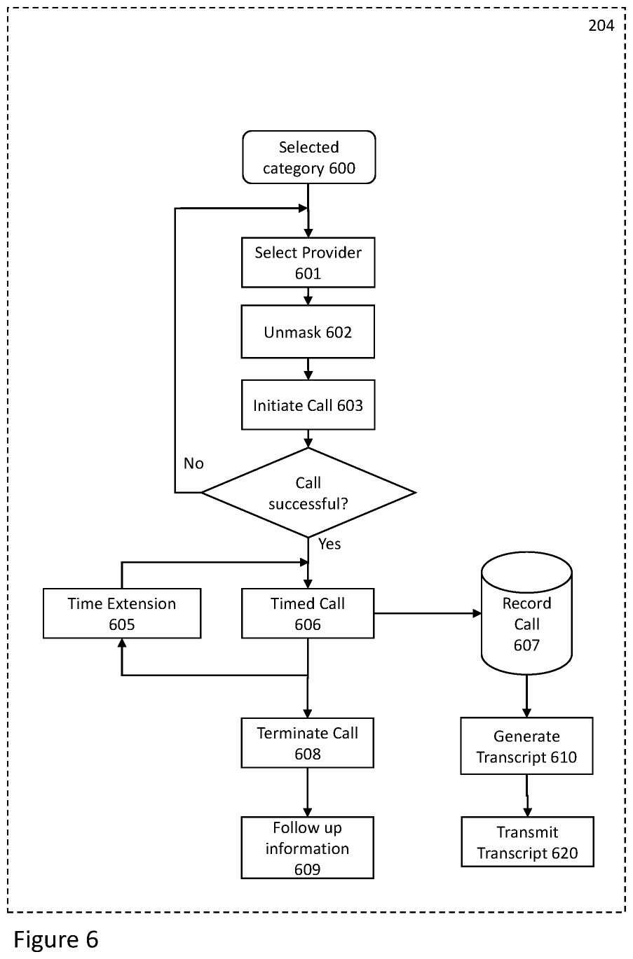

Call processing 204 is further described in FIG. 6. Selected category 600 is obtained from request input 202 and is routed to select provider 601 to select a specific (see above for details). Unmask step 602 transfer consumer identification information such as name, handle, IP address, etc. to on service provider computer system 102. Conversely service provider identification information such as name, handle, IP address, qualifications, ratings, etc. are transferred to consumer computer system 101. Optionally consumer computer system 101 displays some or all of the service provider identification information on consumer computer system 101. Similarly some or all of the consumer identification information is displayed on service provider computer system 102.

Initiate call step 603 initiates a bi-directional audio/video feed (call) between consumer computer system 101 and service provider computer system 102. In a preferred embodiment, the call may either be performed directly between the two computer systems or maybe done indirectly by using server computer system 103 as a proxy or relay agent. It should be noted that during the initiation of the call before it is connected both the consumer 105 and service provider 106 independently have the ability to cancel the call possibly based on the identification information displayed by unmask step 602. Additionally, the system times the duration of the call initiation (i.e., before the provider answers the call) and will automatically cancel the call if not answered in a pre-determined time, e.g., 30 seconds, one minute, etc.

If the call fails either due to cancellation by service provider 106 or the expiration of the pre-determined time or for any other reason (potentially network failure, etc.) then the system returns to select provider 601 to attempt to call again. In a preferred embodiment, the system may alter the prioritize step 504 so as to avoid selecting the same provider as on the previous iteration.

Once the call is successfully initiated (i.e., the service provider answers the call) the system enters step timed call 606 wherein the system times the duration of the call. In a preferred embodiment, calls are limited to a pre-determined duration (e.g., five minutes, seven minutes, etc.) which can be extended by consumer 105 or by service provider 106 via step time extension 605 which typically requires the transfer of some amount of money for some additional pre-determined amount of time. Timed call 606 may provide a video and/or audio indication of the impending termination of the call in order to allow sufficient time for consumer 105 or service provider 106 to exercise time extension 605 before the call is terminated. If time extension 605 is not entered, then upon expiration of the pre-determined amount of the time system terminates the call.

Regardless of how the call terminates (i.e., time expiration or manual disconnection by either consumer 105 or service provider 106) terminate call step 608 is entered which performs logging and other housekeeping functions. In a preferred embodiment, the linking of consumer 105 and service provider 106 is recorded so that the system is able to adjust the priority of this service provider so that the client will be more likely to connect to other service providers in future calls.

Follow up information 609 allows the collection and possible transmission of information after the call has terminated. In a preferred embodiment, consumer 105 may be prompted to provide feedback. Feedback may include information for transmittal to service provider 106, e.g., text messages, World Wide Web links (URLs), voice messages, pictures, etc. For example, a consumer may wish to transmit a text message reminding the service provider of something that was discussed (e.g., “Please don't forget to send me that article you talked about.”). Other feedback may be also be collected, e.g., provider ratings, call quality ratings, overall experience ratings, category matching quality ratings, etc. Some of this other feedback may be used by the system to adjust internal provider prioritization (i.e., prioritize 504) and could be stored in provider database 330. Some of this other feedback could be used to adjust and improve the algorithms of match categories 205 (i.e., named entity recognition 306, model 311, etc.). Similarly service provider 106 may also provide feedback, some of which could be transmitted to consumer 105 (text messages, files, World Wide Web links (URLs), pictures, voice messages, pictures, etc.) and others of which can adjust and improve internal algorithms, e.g., prioritize 504, named entity recognition 306, model 311, etc.

Optionally, record call 607 will record the bi-direction audio/video feed, which is then transcribed by generate transcript 610 and sent to consumer 105 and/or service provider 106 via transmit transcript 620. Generate transcript 610 may also process the video using well known machine learning techniques to further augment the transcription.

### Discover Categories (210)

Before consumer 105 has entered a search query or if the search query is empty or null the system creates a set of categories designated discover categories 210. In some embodiments this may simply be a fixed list of categories, possibly obtained from service computer system 103. In an alternate embodiment, discover categories 210 could be generated dynamically by considering information such as the identity of consumer 105, previous interactions of the system with consumer 105 (e.g., previous search query terms), geographic considerations, availability of service providers, time of day, language of consumer 105, a social media posting of the consumer, a website of the consumer, a publication of the consumer, the consumer's pictures on a social media site or client device, etc.

### Status Changed

The system is monitoring the information that is used either directly or indirectly by match categories 205 and render categories 201 (as described above). Some or all of this information may change due to various events such as initiation or termination of a call, enrollment of a service provider, acquisition of a new category by an existing service provider, changes in corpus topics 307 and/or corpus text 309, changes in spot database 305, changes in service provider availability, etc. The request input step 202 recognizes when the currently displayed information may no longer be correct (e.g., number of available providers is affected by call initiations and terminations, etc.) and exits step 202 looping back to step 201 to generate a new display which is derived from the updated information.

### Service Provider Availability

The availability of a service provider is derived from multiple pieces on information in the system. A service provider may construct and maintain a calendar of availability which is stored in provider database 330 (e.g., Tuesdays from 1-5 pm). A service provider may provide temporary exceptions to the calendar by using service provider computer system 102 (i.e., manually indicating availability or unavailability). The system is also aware of when a service provider is currently participating in a call and can thus automatically determine that the service provider is unavailable. The dynamic behavior of a service provider can also be used by the system, for example, if a service provider has recently failed to answer one or more calls within the pre-determined amount of time, then the service provider could be considered as unavailable possibly for some duration or until the service provider explicitly notifies the system of re-availability.

### Appointment Scheduling (211)

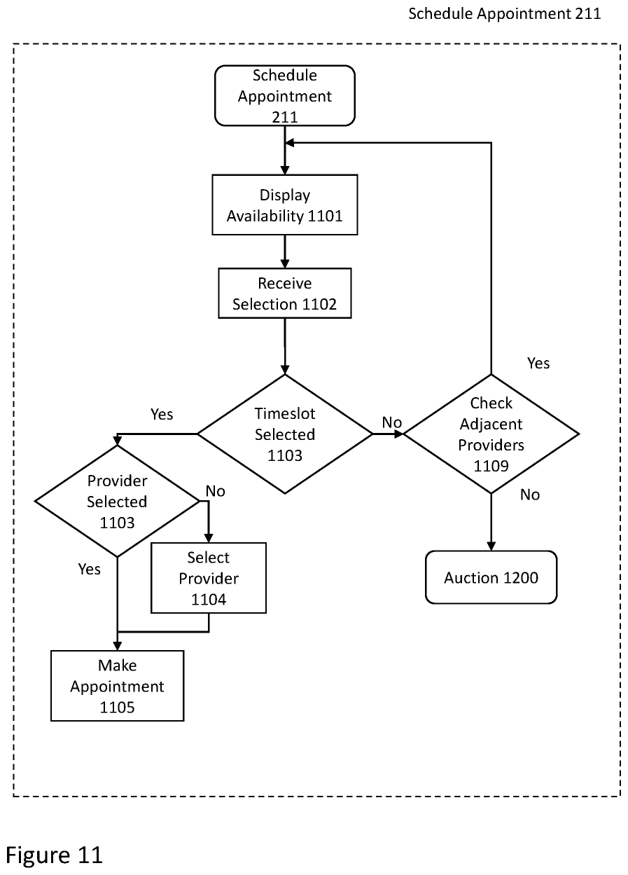

Schedule Appointment 211 is invoked when the one or more service providers of the selected service category have no current availability (see FIG. 11 for details). In Display Availability 1101 the future availability is determined and displayed to the consumer (including the case of no future availability). In one embodiment, the displayed future availability is an aggregate of availability of the one or more service providers. In an alternate embodiment or optionally, the future availability of each of the one or more service providers is displayed. In Receive Selection 1102, the consumer indicates his selection to the system.

Timeslot Selected 1103 determines if the consumer has selected one of the offered future available timeslots. If yes, then Provider Selected 1103 determines if the consumer has selected a specific service provider or has indicated generic acceptance. If a specific provider is selected, then Make Appointment 1105 schedules a future appointment of the consumer and specified service provider at the selected future timeslot using any well-known calendaring technique. In the generic acceptance case, Select Provider 1104 selects a specific provider that has indicated availability at the timeslot selected by the consumer. Select Provider 1104 may use substantially the same criteria and methodology for selecting amongst the one or more service providers with availability at the selected timeslot as was used in Prioritize 504 and Select Provider 505. Once the specific provider is selected then Make Appointment 1105 schedules a future appointment of the consumer and the selected service provider at the selected future timeslot using any well-known calendaring technique.

Timeslot Select 1103 may determine that the consumer has indicated a desire for a timeslot other than one of the displayed timeslots, i.e., where none of the one or more service providers has indicated future availability. In one embodiment the system will attempt to select additional providers with availability at the desired timeslot. In this case Check Adjacent Providers 1109 substantially repeats Match Categories 205 and Render Categories 201 except that in step Select Categories 403 the criteria for selection is relaxed, e.g., a lower quality of match score may be considered acceptable.

If no additional providers can be selected or the consumer rejects the additional offered service providers, the system may attempt to entice a service provider to create additional availability through Auction 1200.

### Auction (1200)

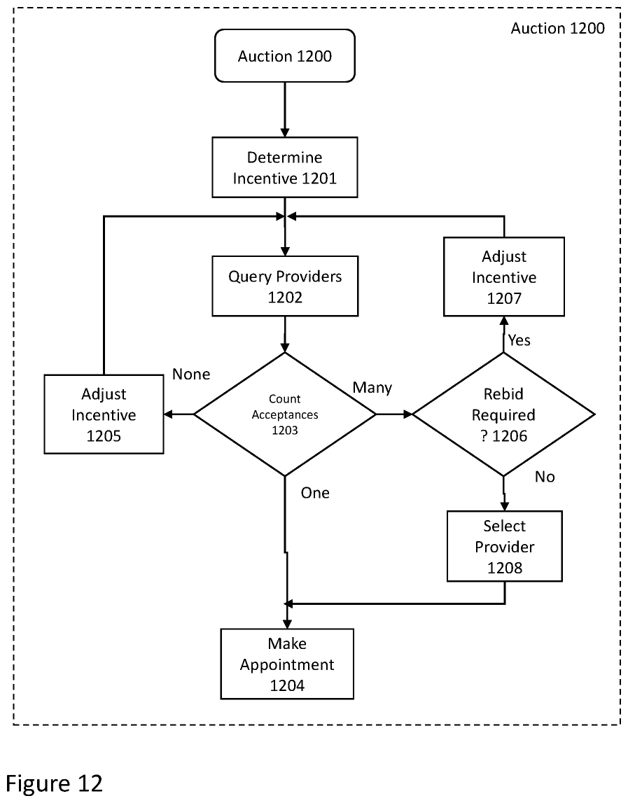

FIG. 12 shows the processing of some embodiments of Auction 1200 (other embodiments are not shown but are clearly described). Auction 1200 intends to entice a service provider to create additional availability at a future timeslot indicated by the consumer. This enticement is presumed to be a financial incentive that is to be determined which could be as low as zero (or even negative). Determine Incentive 1201 determines an initial incentive. In one embodiment, the consumer proposes an initial value which is used to determine the incentive. In another embodiment, the system may use a fixed value or formula to determine the initial incentive based on factors such as: timeslot, provider skill level, number of providers, etc.

Query Providers 1202 offers the incentive to the one or more service providers of the selected category to create availability at the timeslot desired by the consumer. The query process could be serial or parallel.

In the serial case, the system determines an order of querying using criteria that are substantially the same as Prioritize 504 and Select Provider 505. Each provider is queried in turn to determine if he/she will accept the offer. If a provider accepts the offer then Query Providers 1202 terminates immediately without querying additional providers as this is unneeded. If all providers decline to accept the offer then the system may choose to adjust the incentive 1205 and repeat the query process (which could again be serial or parallel).

In the parallel case, the providers are queried in parallel. One form of the parallel query could be in the form of a push notification, i.e., the system sends a notification of the offer. Another form of a parallel query would be a listing of outstanding offers that providers could examine (like a tote board). The number of acceptances of the offer is examined (typically after a period of time has elapsed or perhaps when a sufficient number of responses has been collected from the service providers, e.g. one positive acceptance also known as first-come-first-served) by Count Acceptances 1203. If there is only one acceptance, then Make Appointment 1204 calendars the appointment using any well-known calendaring technique. If there are no acceptances, then the system may choose to increase the incentive and repeat the query (Adjust Incentive 1205, Query Providers 1202). If there are multiple acceptances, then the system may choose to either pick one of the acceptances or initiate a rebid with an adjusted (decreased) incentive. The choice of which service provider acceptance could be made by the system (Select Provider 1208) using criteria substantially similar to Prioritize 504 and Select Provider 505 or, in some embodiments, the consumer could be allowed to make that choice.

In an alternative embodiment, the auction process could reverse the determination of the incentive, i.e., instead of the system proposing an incentive individual, individual providers could propose an incentive level at which they would accept the offer.

In all cases and embodiments, the incentive negotiated between the system and provider is unrelated to any additional charges that might be made to the consumer for the request to create future availability. The system could choose to charge the consumer more or less than the accepted incentive at its discretion.

In all cases and embodiments, the incentive offered to one service provider may not be the same as another service provider. The system may choose increase or decrease the incentive of an individual offer based on criteria substantially similar to those used by Prioritize 504 and Select Provider 505, i.e., more desirable providers may be offered more incentive.

The subject technology is illustrated, for example, according to various aspects described below. These are provided as examples and do not limit the subject technology. It is noted that any of the embodiments may be combined in any combination and placed into a respective independent embodiment. The following is a non-limiting summary of some examples presented herein:

A method to generate live bi-directional video/audio feed between a consumer and a service provider, may include providing a database of service categories, each service category including a listing of one or more service providers that provide services during live bi-directional video/audio feeds related to the service category in which the one or more service providers is included. Additionally or alternatively, in some embodiments the method may include selecting, from the database of service categories, a plurality of service categories for a consumer that are specific to the consumer.

Additionally or alternatively, in some embodiments the selecting may include scraping consumer data from one or more of: a social media posting of the consumer, a website of the consumer, the consumer's pictures on a social media site or client device, a location of the consumer, a preferred language of the consumer, and metadata; analyzing the consumer data to correlate a relationship between the consumer data and the database of service categories, and based on the correlated relationship being above a threshold correlation, selecting the plurality of service categories for the consumer.

In these or other embodiments, service provider data may be scraped from one or more of: a social media posting of the service provider, a website of the service provider, a resume or other document associated with the service provider, pictures from the service provider's social media site or service provider's client device, a location of the service provider, a preferred language of the service provider; analyzing the service provider data to correlate a relationship between the service provider data and the database of service categories, and based on the correlated relationship being above a threshold correlation, selecting the plurality of service categories for the consumer.

Additionally or alternatively, in some embodiments the method may include determining an availability of the service provider to engage the consumer either currently or in the future. In some embodiments, a service provider provides a calendar of future availability (e.g., every Tuesday from 1-5 pm) which may be augmented or superseded by additional, possibly temporary, availability indications (e.g., I'm available now or I'm not available now). The system also knows the unavailability of a provider if he/she is known to be currently connected to a different consumer.

Additionally or alternatively, in some embodiments the method may include providing for rendering, at a client device of the consumer, the selected plurality of service categories.

Additionally or alternatively, in some embodiments the method may include providing for rendering with each service category of the plurality of service categories, at the client device, a real-time status indicator based on the received availability inputs, the real-time status indicator for each service category indicating to the consumer that either: at least one service provider is available to engage the consumer at a current time (e.g., indicated by a green dot) or no service provider is available to engage the consumer at the current time (e.g., indicated by a red dot).

Additionally or alternatively, in some embodiments the method may include managing in real time the availability of the service providers. In these or other embodiments, the managing may include determining whether at least one service provider in each service category is available to engage the consumer, wherein the determining includes counting a number of service providers that currently indicate availability to engage the consumer.

Additionally or alternatively, the managing in some embodiments may include in response to the determining, updating the real-time status indicator of each service category to reflect a current availability of one or more service providers in the service category by re-rendering at the client device the corresponding real-time status indicator, wherein a status change for any service category constitutes a change in availability to engage the consumer as follows: (i) changing from at least one available service provider in the service category to no service provider in the in the service category is currently available, or (ii) changing from no service provider available to at least one available service provider in the service category.

Additionally or alternatively, in some embodiments the method may include obtaining a consumer input that includes a selection of a service category among the selected plurality of service categories that has a real-time status indicator indicating at least one service provider is available to engage the consumer, the consumer input effective to initiate within a time window a live video/audio feed between the consumer and one of the one or more service providers of the selected service category.

Additionally or alternatively, in some embodiments the method may include upon the initiating and within the time window, linking the consumer to one of the one or more service providers of the selected service category.

Additionally or alternatively, in some embodiments the method may include determining which of the one or more service providers to link with the consumer based on one or more of the scraped consumer data, a search query, the availability of the one or more service providers between a most recent re-rendering of the real-time status indicator and receiving the consumer input, and a priority relationship among the one or more service providers.

In some embodiments, the availability inputs from the one or more service providers received from the one or more service providers between a most recent re-rendering of the real-time status indicator and receiving the consumer input changes which service providers are available to engage the consumer such that either more or fewer service providers are actually available to engage the consumer.

In some embodiments, the priority relationship among the one or more service providers determines an order to which service providers are linked with consumers, the priority relationship based on one or more of consumer ratings of the service provider, a subscription tier paid by the service provider that includes a certain order or threshold ranking, and a quality control assessment of the service provider that includes a skill ranking within the service category.

Additionally or alternatively, in some embodiments the method may include in response to the determination, establishing an internet protocol (IP) packet-based communication connection, including a video and/or audio communication connection (e.g., for bi-directional video and/or audio), between the client device of the consumer and a device of the service provider determined to link to the client device, the IP packet-based transmission connection established by a system that hosts the database of service categories, wherein the system includes the IP addresses of the client device and the device of the service provider to establish the communication connection and the client device and the device of the service provider are unware of each other's IP addresses before the determination of the service provider.

Additionally or alternatively, in some embodiments the method may include unmasking the consumer and the service provider, wherein the consumer and the service provider are masked entities unknown to each other at the time the consumer selects the service category, and wherein unmasking includes: retrieving identifying data of the consumer that corresponds to de-identified data of the consumer, retrieving identifying data of the service provider that corresponds to de-identified data of the service provider, and sending identifying data of both the consumer and the service provider to the client device of the consumer and the client device of the service provider via the established IP packet-based transmission connection.

Additionally or alternatively, in some embodiments the method may include after linking the consumer to one of the one or more service providers, generating a live bi-directional video/audio feed between the consumer and the one or more service providers for a predetermined duration of time to allow the service provider to provide the respective services, wherein upon expiration of the duration the live bi-directional video/audio feed is terminated.

In other embodiments, a method to generate live bi-directional video/audio feed (e.g., bi-directional video and/or audio) between a consumer and a service provider, may include
providing a database of service categories, each service category including a listing of one or more service providers that provide services during live bi-directional video/audio feeds related to the service category in which the one or more service providers is included;
selecting, from the database of service categories, a plurality of service categories for a consumer that are specific to the consumer,
wherein the selecting includes:
scraping consumer data from one or more of: a social media posting of the consumer, a website of the consumer, pictures from the consumer's social media site or client device, a location of the consumer, and a preferred language of the consumer,
analyzing the consumer data to correlate a relationship between the consumer data and the database of service categories, and
based on the correlated relationship being above a threshold correlation, selecting the plurality of service categories for the consumer;
receiving an availability input from a service provider, the availability input from the service provider indicating an availability of the service provider to engage the consumer;
providing for rendering, at a client device of the consumer, the selected plurality of service categories;
providing for rendering with each service category of the plurality of service categories, at the client device, a real-time status indicator based on the received availability inputs, the real-time status indicator for each service category indicating to the consumer that either: at least one service provider is available to engage the consumer at a current time or no service provider is available to engage the consumer at the current time;
managing in real time the availability of the service providers, the managing including:
determining whether at least one service provider in each service category has sent an availability input indicating that the service provider is available to engage the consumer, wherein the determining includes counting a number of service providers that currently indicate availability to engage the consumer;
in response to the determining, updating the real-time status indicator of each service category to reflect a current availability of one or more service providers in the service category by re-rendering at the client device the corresponding real-time status indicator, wherein a status change for any service category constitutes a change in availability to engage the consumer as follows: (i) changing from at least one currently available service provider in the service category to no service provider in the in the service category currently available, or (ii) changing from no service provider currently available to at least one service provider in the service category currently available;
obtaining a consumer input that includes a selection of a service category among the selected plurality of service categories that has a real-time status indicator indicating at least one service provider is available to engage the consumer, the consumer input effective to initiate within a time window a live bi-directional video/audio feed between the consumer and one of the one or more service providers of the selected service category;
upon the initiating and within the time window, linking the consumer to one of the one or more service providers of the selected service category, the linking including:
determining which of the one or more service providers to link with the consumer based on one or more of the scraped consumer data, a search query, the availability inputs received from the one or more service providers between a most recent re-rendering of the real-time status indicator and receiving the consumer input, and a priority relationship among the one or more service providers,
wherein the availability inputs from the one or more service providers received from the one or more service providers between a most recent re-rendering of the real-time status indicator and receiving the consumer input changes which service providers are available to engage the consumer such that either more or fewer service providers are actually available to engage the consumer, and
wherein the priority relationship among the one or more service providers determines an order to which service providers are linked with consumers, the priority relationship based on one or more of consumer ratings of the service provider, a subscription tier paid by the service provider that includes a certain order or threshold ranking, and a quality control assessment of the service provider that includes a skill ranking within the service category;
in response to the determination, establishing an internet protocol (IP) packet-based communication connection, including a video and/or audio communication connection, between the client device of the consumer and a device of the service provider determined to link to the client device, the IP packet based transmission connection established by a system that hosts the database of service categories, wherein the system includes the IP addresses of the client device and the device of the service provider to establish the communication connection and the client device and the device of the service provider are unware of each other's IP addresses before the determination of the service provider; and
unmasking the consumer and the service provider, wherein the consumer and the service provider are masked entities unknown to each other at the time the consumer selects the service category, and wherein unmasking includes: retrieving identifying data of the consumer that corresponds to de-identified data of the consumer, retrieving identifying data of the service provider that corresponds to de-identified data of the service provider, and sending identifying data of both the consumer and the service provider to the client device of the consumer and the client device of the service provider via the established IP packet-based transmission connection; and
after linking the consumer to one of the one or more service providers, generating a live bi-directional video/audio feed between the consumer and the one or more service providers for a predetermined duration of time to allow the service provider to provide the respective services, wherein upon expiration of the duration the live bi-directional video/audio feed is terminated.

Modifications, additions, or omissions may be made to any portion of the disclosure (such as the example embodiments outlined above) without departing from the scope of the present disclosure. For example, it is understood that, for this and other processes, operations, and methods disclosed in this disclosure, the functions and/or operations performed may be implemented in differing order. Furthermore, the outlined functions and operations are only provided as examples, and some of the functions and operations may be optional, combined into fewer functions and operations, or expanded into additional functions and operations without detracting from the essence of the disclosed embodiments.

As indicated above, the embodiments described in this disclosure may include the use of a special purpose or general purpose computer (e.g., processor 110) including various computer hardware or software modules, as discussed in greater detail below. Further, as indicated above, embodiments described in this disclosure may be implemented using computer-readable media (e.g., memory 112) for carrying or having computer-executable instructions or data structures stored thereon.

In some embodiments, the different components, modules, engines, and services described in this disclosure may be implemented as objects or processes that execute on a computing system. While some of the systems and methods described in this disclosure are generally described as being implemented in software (stored on and/or executed by general purpose hardware), specific hardware implementations or a combination of software and specific hardware implementations are also possible and contemplated.

In accordance with common practice, the various features illustrated in the drawings may not be drawn to scale. The illustrations presented in the present disclosure are not meant to be actual views of any particular apparatus (e.g., device, system, etc.) or method, but are merely idealized representations that are employed to describe various embodiments of the disclosure. In addition, some of the drawings may be simplified for clarity. Thus, the drawings may not depict all of the components of a given apparatus (e.g., device) or all operations of a particular method.

Terms used in this disclosure and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, it is understood that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

Additionally, the use of the terms “first,” “second,” “third,” etc., are not necessarily used in this disclosure to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited in this disclosure are intended for pedagogical objects to aid the reader in understanding the invention and the concepts contributed by the inventor to furthering the art, and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

1. A method to schedule a live bi-directional video/audio feed between a consumer and a service provider comprising:
obtaining a consumer query;
analyzing the consumer query to generate one or more service categories each service category including one or more service providers that provide services to the consumer responsive to the query;
displaying to the consumer the one or more service categories;
receiving a consumer input to select a service category from the one or more service categories;
determining the current and future availabilities of each of the one or more service providers;
displaying to the consumer current and future availabilities of the one or more service providers;
receiving a consumer input to select one of the availabilities; and
initiating a live bi-directional video/audio feed responsive to the selected availability.

2. The method of claim 1, wherein the displaying to the consumer the current and future availabilities of the one or more service providers further comprises:
aggregating the availability of the one or more service providers; and
displaying to the consumer the aggregated availabilities.

3. The method of claim 1, wherein the displaying to the consumer the current and future availabilities of the one or more service providers further comprises:
displaying the current and future availability of each of the one or more service providers.

4. The method of claims 1, 2, or 3 wherein there is no current availability of any of the one or more service providers.

5. A method to schedule an appointment for a live bi-directional video/audio feed between a consumer and a service provider comprising:
obtaining a consumer query;
analyzing the consumer query to generate one or more service categories each service category including one or more service providers that provide services to the consumer responsive to the query;
displaying to the consumer the one or more service categories;
receiving a consumer input to select a service category from the one or more service categories;
determining the current and future availabilities of each of the one or more service providers;
displaying to the consumer the current and future availabilities of the one or more service providers;
receiving a consumer input indicating none of the current and future availabilities is acceptable.

6. The method of claim 5 wherein the receiving a consumer input indicating none of the current and future availabilities is acceptable further includes:
a consumer input indicating a desire for one or more alternative availabilities.

7. The method of claim 6 wherein the consumer input indicating a desire for one or more alternative availabilities further includes:
a financial incentive.

8. The method of claims 6 or 7 further comprising:
querying each of the one or more service providers, wherein the query includes the desired alternative availabilities.
